(19) United States
(12) Patent Application Publication
(10) Pub. No.: US 2025/0216240 A1
(43) Pub. Date: Jul. 3, 2025

(54) INTELLIGENT SYSTEM FOR REAL-TIME MEASUREMENTS AND ANALYSIS OF FUEL OILS, FOR QUANTITATIVE AND QUALITATIVE ASSESSMENT AND ACCEPTANCE

(71) Applicant: ENSOMATOSYS GROUP LTD., Limassol (CY)

(72) Inventors: Dimitrios CHASAMPALIS, Limassol (CY); Dimitrios KONTAXIS, Neos Kosmos Athens (GR); Ioannis VAVOURAKIS, Athens (GR)

(73) Assignee: ENSOMATOSYS GROUP LTD., Limassol (CY)

(21) Appl. No.: 18/805,469

(22) PCT Filed: Feb. 16, 2022
(86) PCT No.: PCT/EP2022/025052

§ 371 Date: Aug. 14, 2024

(51) Int. Cl. G01F 15/063; B63B 25/12; B63B 79/10; G01F 1/667; G01N 29/02; G01N 29/44; G01N 33/22

(52) U.S. Cl. CPC G01F 15/063; B63B 25/12; B63B 79/10; G01F 1/667; G01N 29/02; G01N 29/44; G01N 33/22

(57) ABSTRACT

A system for real-time measurements and analysis of fuel oils and the quantitative and qualitative assessment and acceptance of the fuel, which is configured to perform the acquisition, monitoring, presentation, analysis and storage of numerous parameters in real time, during a ship's refueling or fuel cargo loading-unloading process, and is implemented using: a) portable device configured to be installed at the leading edge of the ship's bunkering or fuel cargo loading-unloading pipe, in line with the fuel flow and including i) employing ultrasound technology and ii) sensors to perform chemical analysis both for quantitative and qualitative assessment and acceptance, i.e. measuring the % content of a plurality of chemical elements in the fuel, b) a cloud application, and c) a mobile application.

### 1. FIELD OF APPLICATION

a. The Invention is mainly applied to the refueling process of ships however, potentially, it can be applied to the refueling process of all means of transport. The international term for the refueling process of ships is “bunkering”. Moreover, the Invention can be applied to the loading or unloading process of fuel oils of any type, used a cargo. The main objectives of the Invention are:
   (1) Delivery—acceptance of the correct quantity of fuel.
   (2) Delivery—acceptance of fuel quality which complies with the ISO 8217: 2017 specifications or any international/national standard in force related to the refueling process or fuel cargo loading-unloading process, thus eliminating or minimizing the cases of adulterated fuel or fuel of bad quality, which may have catastrophic consequences for the ship's/mean's engines and/or the environment.
   (3) Optimal management of the refueling/bunkering or fuel cargo loading-unloading process and minimization (or even elimination) of the economic losses.
b. The Invention is a System which achieves the abovementioned objectives by collecting, monitoring, presenting and analyzing in real time numerous parameters of interest, during refueling/bunkering or fuel cargo loading-unloading process, by exploiting at least the following sub-systems:
   (1) The Portable Device.
   (2) The Cloud Application and Data Base (DB).
   (3) The Mobile Application (for smartphones or any portable device with short range wireless communication capability).

### 2. DESCRIPTION OF THE PROBLEM AND THE NECESSITY OF THE INVENTION

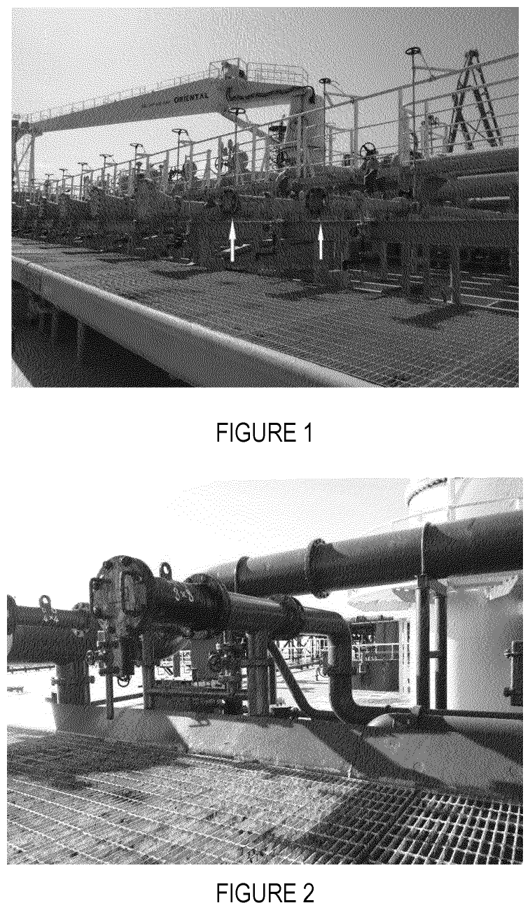

The process described below is focused but not limited to the bunkering procedures (i.e. it can be generalized to the fuel cargo loading-unloading process, refueling process of all means of transport, etc.).
a. The bunkering process of a ship involves the following steps-procedures:
   (1) Step 1: A floating fuel tank (vessel) approaches the ship to be refueled. The term used internationally for this tank is “barge”.
   (2) Step 2: The barge connects its pipe to the ship's refueling pipe. The connection can be made either directly, if the cross section of the two pipes is the same, or through a “cross section converter” (referred to in shipping as “reducer”), if the cross section of the two pipes is different. The connection is made with flanges and bolts, as shown in FIGS. 1 and 2.
   (3) Step 3: A fuel sample collection assembly is fit/installed at the connection point between the aforementioned pipes (see Step 2). At the end of the bunkering process, the collected fuel is divided into four (4) equal samples/parts, as follows:
      (a) Two parts remain on board.
      (b) One part is delivered to the barge.
      (c) One part is sent by the ship-owning company to a third-party laboratory, which carries out a chemical analysis.
   (4) Step 4: The bunkering process starts with specific pressure (measured in bars) and fuel flow rate (measured in m³/h). The fuel temperature during bunkering must allow for smooth fuel flow. Therefore, fuels of high viscosity (measured in centistokes (cSt) shall be supplied (from the barge) at a relatively high temperature (e.g. 40° C.).
   (5) Step 5: The bunkering process ends and quantitative acceptance of the fuel follows: the ship's engineers, measure (manually) the level/height of the fuel in the ship's tank, which corresponds to a specific volume of fuel (based on the knowledge of the tanks dimensions). This procedure is followed in all cases.
b. The qualitative acceptance of the fuel is notified after several days, after the completion of the chemical analysis by the laboratory (see Step 3/c, above).
c. The main problems of the above process are the following:
   (1) Regarding the quantitative acceptance:
      (a) The procedure of measuring the “height” of fuel inside the ship's tank is not commonly accepted. The barge usually expresses objections for the accuracy of the measurement performed at the ship side, while its measurements (made with “positive displacement” or “coriolis” flowmeters) are considered, according to the international practice, the most valid ones. In these cases, which tend to be the rule, the solution is given as follows:
         1/ either by mutual compromise, leading one of the two parties (the ship or the barge) to significant economic losses,
         2/ or at the court, which results in court decisions which lead again one of the two parties to significant economic losses.
      (b) In case of adulteration of the supplied fuel with air bubbles (from the barge), the “height” of the fuel inside the tank of the ship is “temporarily” correct. After some period of time (and after the barge has departed and the quantitative acceptance of the fuel has taken place) the air bubbles escape from the fuel and hence, its level inside the tank decreases dramatically. However, the ship-owning company, after the departure of the barge, cannot prove the fraud which leads to significant economic losses. The adulteration of the supplied fuel with air bubbles is referred to in shipping as “cappuccino effect”.
   (2) Regarding the qualitative acceptance, the chemical analysis of the fuel carried out by the laboratory, as described in paragraph 2a (3) (c) above, is always delayed for several days. In the meantime, the ship moves/travels with the fuel it received. In case the fuel quality is improper (e.g. high percentage of water or chemical elements out of limits, which cause problems in the operation of the engines), this will affect the operation, efficiency and service life of the ship's engines, with a major mid-/long-term economic impact on the ship-owning company. In case the supplied fuel is of very bad quality and causes immediate and serious problems in the operation of the engines, the ship shall interrupt its voyage and unload the supplied fuel immediately, a process with very high cost for the ship-owning company.
d. The added value of the present Invention lies at least in the following:
   (1) It is the only known portable System able to perform measurements and chemical analysis in real time, during bunkering, both for quantitative and qualitative assessment and acceptance.
   (2) The measurements and data are available in real-time in a Cloud or a Mobile Application.
   (3) To the best of the inventors' knowledge, there is no other similar System used for the bunkering process. Simple flowmeters cannot be considered as “similar systems”, since:
      (a) They are not portable.
      (b) In most cases, they do not transmit data and do not provide notifications and/or alarms.
      (c) They do not perform chemical analysis.
   (4) Through the implemented chemical analysis, it can provide to the ship's personnel and to the ship-owning company direct/real time information about the quality of the received fuel and allows for an immediate decision for the cessation of the bunkering process, in case the fuel quality is out of the ISO 8217 specifications.
   (5) It can put in for a commonly accepted System that will minimize or eliminate:
      (a) Phenomena of fraud.
      (b) Economic losses due to:
         1/ Delivery of less (than paid) amount of fuel.
         2/ Low efficiency of ship's engines.
         3 /Damage of ship's engines.
         4/ Necessity for fuel unloading.
         5/ Court costs.

### 3. ANALYSIS OF THE TECHNO-ECONOMIC OBJECTIVES OF THE INVENTION

As an indicative example or case study for the usefulness of the Invention, the following incident can be mentioned (without any reference to the names of the involved companies), which has been recorded in shipping and is related to the bunkering process:

In 2011, a ship received 410 tons of fuel, type IFO-180cSt, in the region of West Africa. Responsible for the delivery of the fuel were a German and a Cypriot company (intermediary suppliers) which had bought the fuel from an African supplier. After the delivery of the fuel, the ship departed, but after 72 hours of travel, problems began to appear in its engines, due to bad fuel quality (as it was proved by the chemical analysis several days later). The need for unloading the fuel from the ship's tanks was imperative. The fuel unloading process took place in a port in Sweden (the only port available for this process, due to the dimensions of the ship). This process resulted in a total economic loss of USD 350,000 for the intermediary suppliers (i.e. the German and the Cypriot company), which were never able to claim the corresponding amount from the African supplier. In addition, the ship suffered serious damages in its engines and delayed its planned voyage for 10 days, with a significant economic impact on the ship-owning company.

Apparently, if there was a system for on-site (real-time) chemical analysis of the fuel, the bunkering process would be ceased from the very first minutes, protecting both the ship-owning company and the intermediary suppliers, from significant economic losses.

Many similar incidents have been reported in shipping. In general, ship-owners' losses in each bunkering are estimated from tens of thousands of USD, mainly due to the delivery of less fuel, to hundreds of thousands of USD, due to “non-ISO 8217” fuel deliveries (which cause damage to the ship's engines), delays, court costs, etc. The losses under consideration are estimated at 4% (at least) of the cost of the supplied fuel at each bunkering.

Therefore, for a company with a fleet of only 10 vessels, with an average fuel capacity of 1000 tn IFO-380cSt per ship, the annual economic losses are calculated as follows:

[Number of Vessels]×[Average Fuel Capacity(tn)]×[Cost/tn(USD)]×[Number of bunkering/Year]×[4% Loss]=10×1000×650×10×4%=$ 2,600,000.00 (Note: the Cost/tn has been considered 650 USD, based on the current fuel oil cost (February 2022)).

Using an intelligent system for real-time quantity and quality measurements/analysis, the ship-owing company would be able to save the aforementioned amount (on an annual basis). In addition, it will be able to protect intermediary suppliers from delivering less and/or unforeseen quality of fuel, which leads them to significant economic losses and loss of their reliability.

In order to achieve the objectives of the Invention, the System meets:
High reliability.
Certifications for use in the marine environment.
Flexible design and ease of use-handling.
Affordable cost.

### 4. SYSTEM DESCRIPTION

The System includes the following configuration items/parts/devices:

### 4.1. Portable Measuring and Chemical Analysis Device

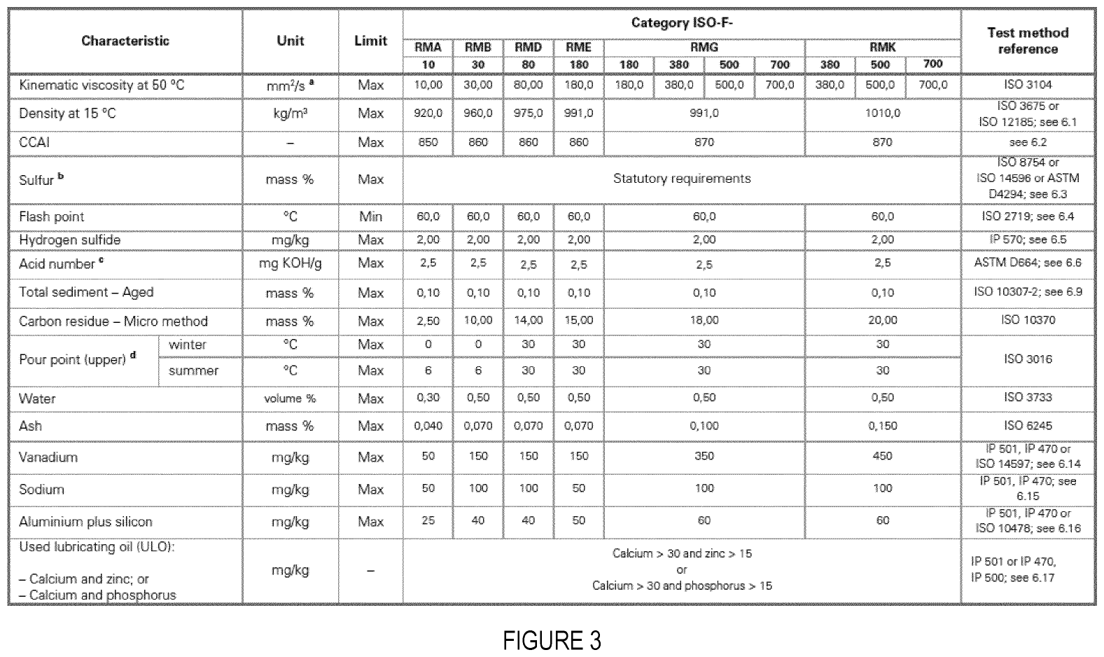

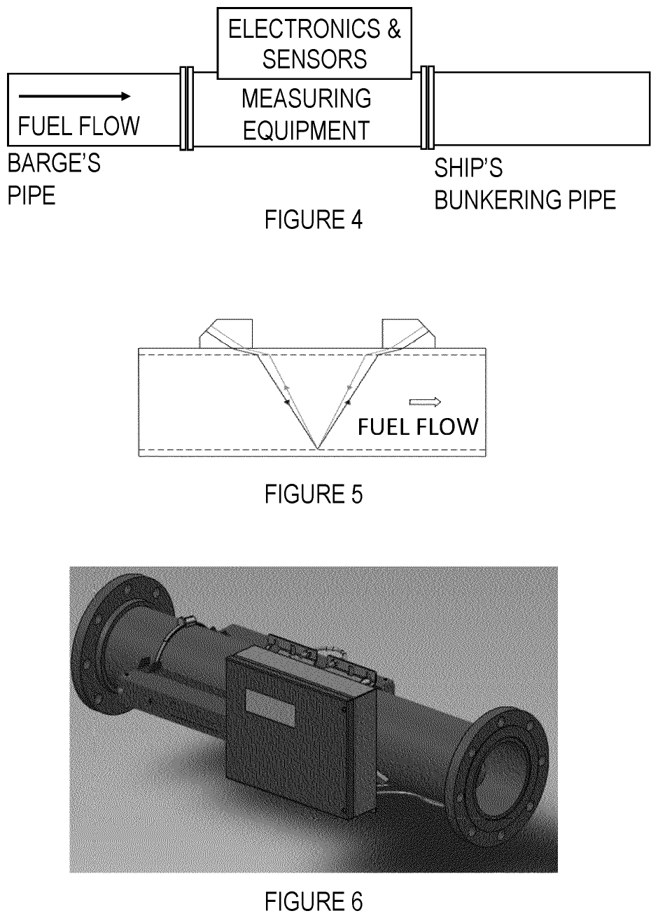

It is a portable sensor platform/device powered by a rechargeable battery. The device has the shape of a pipe and is adapted/installed at the leading edge of the ship's bunkering or fuel cargo loading-unloading pipe, in line with the fuel flow, as shown in FIG. 4. The device does not interrupt or obstruct the fuel flow during the bunkering process. However, the device can also be a “fixed installation” on the ship's piping system (in line with the fuel flow) and be power supplied permanently by a DC/DC or AC/DC adapter. The device performs at least the following functions simultaneously and in real time:
a. Provides measurements of numerous parameters related to the quantitative acceptance of the fuel. Indicative parameters are listed below:
   (1) Operating and standard (normalized to any desired temperature) fuel volume rate (m³/h).
   (2) Operating and standard (normalized to any desired temperature) fuel mass rate (tn/h).
   (3) Operating and standard (normalized to any desired temperature) total fuel volume (m³).
   (4) Operating and standard (normalized to any desired temperature) total fuel mass (tn).
b. Identifies the category/type of the fuel (e.g. RMG 380, RMG 180, Gasoil, etc.).
c. Detects possible adulteration of the fuel with air bubbles (providing alarms when the detected level exceeds a desired limit).
d. Performs instant chemical analysis of the fuel and measures:
   (1) Fuel kinematic and dynamic viscosity (cSt).
   (2) Fuel operating and standard (normalized to any desired temperature) density (kg/m³) and API degree.
   (3) Fuel temperature (° C.).
   (4) Fuel humidity (% water content (H₂O)).
   (5) % content of various chemical elements in the fuel such as S, Al, Si, Ca, V, P, Zn, Na, etc. (all chemical elements of the “Periodic Table” can be detected, according to the needs of the application).
e. Detects the location of the bunkering site (WGS84 coordinates), exploiting all available Global Navigation Satellite Systems (GPS, Glonass, Beidou, Galileo).
f. Provides notifications and alarms in case any of the parameters (of paragraphs 4.1.a.-d.) exceed specific limits/thresholds, as defined in the ISO 8217: 2017 (Petroleum products—Fuels (class F)—Specifications of marine fuels) or any international/national standard in force related to the refueling or fuel cargo loading-unloading process, as shown in FIG. 3. Regarding location (paragraph 4.1.e), geo-fencing notifications and alarms can be provided. These limits/thresholds can be set/configured dynamically and remotely by the System User, in the following ways:
   (1) From a Cloud Application, via the cellular network (using any available cellular technology) or satellite communications or any radio link for communication with a local router/gateway (like, but not limited to, WiFi, WiMax, LoRa, LoRaWAN, Zigbee, Bluetooth, Z-Wave, Sigfox, etc.).
   (2) From a Mobile Application, via a short-range radio link (using any short-range radio link available on Mobile Phones).
g. Implements the measurements of paragraphs 4.1.a.-e. with a remotely configurable sampling rate and transmits all bunkering/fuel cargo data:
   (1) To a Cloud Application, via the cellular network (using any available cellular technology) or satellite communications or any radio link for communication with a local router/gateway (like, but not limited to, WiFi, WiMax, LoRa, LoRaWAN, Zigbee, Bluetooth, Z-Wave, Sigfox, etc.).
   (2) To a Mobile Application, via a short-range radio link (using any short-range radio link available on Mobile Phones).
i. Stores locally all the above mentioned data.

For the implementation of the functions of paragraphs 4.1.a.-c the Portable Device employs ultrasound (u/s) technology, based on a commercial solution, specially configured and customized for the needs of the Invention. More specifically, an audio source generates and transmits periodic sound signals—pulses of appropriate frequency (Hz) which pass through the fuel and are received by a u/s receiver. This is done in the direction of the fuel flow, as well as in the opposite direction, as shown in FIG. 5. The system can measure the propagation delay of the sound signals between the transmitter and the receiver, which is affected by the flow rate, the density (or the API degree) and the temperature of the fuel. Neither the u/s transmitter nor the receiver are in contact with the fuel, as shown in FIG. 5. The measured propagation delay of the sound signals corresponds to a specific combination of density (or API degree) and fuel speed. Temperature measurements are also taken into account and are performed using specialized temperature probes. Knowing the density, the speed and the temperature of the fuel, as well as the dimensions of the Portable Device, the device calculates the parameters of paragraphs 4.1.a.-b., as well as the presence of air bubbles in the fuel (as described in 4.1.c), which “distort” the fuel flow and affect various diagnostic parameters monitored (in real time) by the processing unit of the device.

For the measurements of paragraphs 4.1.d.-e., the Portable Device employs various sensors of appropriate technology. All device functions, such as
real time execution of measurements,
real time analysis, processing and storage of measurements,
real time transmission of measurements, notifications and alarms,
remote configuration and Firmware (FW) updates,
   are implemented by a suitable ECU (Electronic Control Unit) which includes:
an embedded processing unit, based on a microprocessor unit and various interfaces,
a cellular transceiver (using any available cellular technology) or a satellite transceiver or any radio link for communication with a local router/gateway (like, but not limited to, WiFi, WiMax, LoRa, LoRaWAN, Zigbee, Bluetooth, Z-Wave, Sigfox, etc.).
a real time clock (RTC).
   An indicative representation of the Portable Device is depicted in FIG. 6.

### 4.2. Cloud Application and Data Base

The Cloud Application and the System's Data Base have been developed in order to:
a. Support all of the functions/features of the Portable Device, as described in detail in paragraph 4.1.
b. Support other functions, as follows:
   (1) Storage and processing of historic data.
   (2) Statistical and economic analysis.
   (3) User management.

### 4.3. Mobile Application

The Mobile Application has been developed in order to support the functions of paragraph 4.1. This Application shall be used on a Mobile Phone in the vicinity of the Portable Device. The Radio Access Technology (RAT) between the Mobile Phone and the Portable Device can be any short-range radio link available on Mobile Phones.

### 5. CONCLUSIONS

The Invention has been designed and a functional prototype has been put under test in certified labs. Results show that the Invention contributes to the following:
a. Delivery/acceptance of the correct quantity of fuel (with tolerance ≤0.5%).
b. Delivery—acceptance of fuel quality which complies with the ISO 8217: 2017 specifications, or any international/national standard in force.
c. Optimal management of the refueling/fuel cargo loading-unloading process and minimization (or even elimination) of the economic losses.

The innovation of the Invention lies in the following:
a. It is the only portable System, which can provide to the ship's personnel and the ship-owning company real time information about the quantity & quality of the received fuel, enabling an early shutdown/cessation of the bunkering/fuel cargo loading-unloading process, in case certain conditions and limits/thresholds are not met. In this context, the Invention could put in for a commonly accepted System, able to minimize or eliminate economic losses and phenomena of fraud.
b. Any refueling/fuel cargo loading-unloading process is supported by a Cloud Application and Data Base, as well as by a Mobile Application, which allow for:
Remote real-time data acquisition.
Remote real-time notifications and alarms.
Remote management, configuration and Firmware updates of the Portable Device.
Data analytics.
Preservation of historic data and statistics.

We claim:

1. A for real-time measurements and analysis of fuel oils for quantitative and qualitative assessment and acceptance of the fuel, which is configured to perform the acquisition, monitoring, presentation, analysis and storage of numerous parameters in real time, during a ship's refueling or fuel cargo loading-unloading process, and is implemented using:
a. a portable device configured to be installed at the leading edge of the ship's bunkering or fuel cargo loading-unloading pipe, in line with the fuel flow and including i) means employing ultrasound technology and ii) sensors to perform chemical analysis both for quantitative and qualitative assessment and acceptance, i.e. measuring the % content of a plurality of chemical elements in the fuel;
b. a cloud application, and
c. a mobile application.

2-4. (canceled)

5. The system according to claim 1, whereby the means employing ultrasound technology are means to i) provide measurements of parameters related to the quantity of the fuel, ii) identify the category/type of the fuel and iii) detect adulteration of the fuel with air bubbles.

6. The system according to claim 1, whereby the portable device has the shape of a pipe.

7. The system according claim 1, whereby the portable device includes an Electronic Control Unit (abb.: “ECU”) including an embedded processing unit, a cellular transceiver or a satellite transceiver or a radio link for communication with a local router/gateway and a real time clock, which ECU is connected with sensors and is configured to transmit measurements stored in the ECU to the cloud application and the mobile application.

8. A method to use a system according to claim 1, comprising the following steps:
i. installing the portable device at the leading edge of the ship's bunkering or fuel cargo loading-unloading pipe, in line with the fuel flow;
ii. measuring parameters related to the quantity of the fuel;
iii. identifying the category/type of the fuel;
iv. detecting adulteration of the fuel with air bubbles if any and providing alarms when the detected level exceeds a desired limit;
v. performing instant chemical analysis of the fuel;
vi. setting threshold limits of any of said parameters, category/type of fuel, adulteration of the fuel with air bubbles, and chemical analysis, and
vii. providing notifications and alarms in case any threshold limit is exceeded.

9. The method according to claim 8, further comprising:
setting threshold limits of any of said parameters, category/type of fuel, adulteration of the fuel with air bubbles, and chemical analysis as defined in ISO 8217:2017.

10. The method according to claim 8, further comprising:
identifying the category/type of the fuel according to ISO 8217:2017.

11. A device for real-time measurements and analysis of fuel oils and the quantitative and qualitative assessment and acceptance of the fuel that is configured to perform the acquisition, monitoring, presentation, analysis and storage of numerous parameters in real time, during a ship's refueling or fuel cargo loading-unloading process, characterized in that:
a. the device is a portable device configured to be installed at the leading edge of the ship's bunkering or fuel cargo loading-unloading pipe, in line with the fuel flow and including means employing ultrasound technology and sensors to perform chemical analysis, i.e. measuring the % content of a plurality of chemical elements in the fuel, and
b. the device has an Electronic Control Unit (abb.: ECU) including an embedded processing unit, a cellular transceiver or a satellite transceiver or a radio link for communication with a local router/gateway and a real time clock, which ECU is connected with sensors and is configured to transmit measurements stored in the ECU to a cloud application and a mobile application.

12. The system according to claim 5, whereby the portable device has the shape of a pipe.

13. The system according to claim 5, whereby the portable device includes an Electronic Control Unit (abb.: “ECU”) including an embedded processing unit, a cellular transceiver or a satellite transceiver or a radio link for communication with a local router/gateway and a real time clock, which ECU is connected with sensors and is configured to transmit measurements stored in the ECU to the cloud application and the mobile application.

14. The system according to claim 6, whereby the portable device includes an electronic control unit (abb.: “ECU”) including an embedded processing unit, a cellular transceiver or a satellite transceiver or a radio link for communication with a local router/gateway and a real time clock, which ECU is connected with sensors and is configured to transmit measurements stored in the ECU to the cloud application and the mobile application.

15. The method according to claim 9, further comprising:
identifying the category/type of the fuel according to ISO 8217:2017.

16. The method of claim 8, further comprising:
employing ultrasound technology to i) provide measurements of parameters related to the quantity of the fuel, ii) to identify the category/type of the fuel and iii) to detect adulteration of the fuel with air bubbles.

17. The method of claim 8, further comprising:
the portable device having the shape of a pipe.

18. The method of claim 8, further comprising:
the portable device including an electronic control unit (abb.: “ECU”) including an embedded processing unit, a cellular transceiver or a satellite transceiver or a radio link for communication with a local router/gateway and a real time clock, which ECU is connected with sensors and is configured to transmit measurements stored in the ECU to the cloud application and the mobile application.
